(19) United States
(12) Patent Application Publication
(10) Pub. No.: US 2021/0100465 A1
(43) Pub. Date: Apr. 8, 2021

(54) HEART RHYTHM PROCESSING METHOD, ELECTRONIC DEVICE, AND COMPUTER PROGRAM

(71) Applicants: Acer Healthcare Inc., NEW TAIPEI CITY (TW); National Taiwan University Hospital, Taipei City (TW)

(72) Inventors: Ke-Han PAN, New Taipei City (TW); Yu-Chun WU, New Taipei City (TW); Yueh-Yarng TSAI, New Taipei City (TW); Pin-Cyuan LIN, New Taipei City (TW); Hsien-Yang CHIANG, New Taipei City (TW); Yen-Bin LIU, Taipei City (TW)

(21) Appl. No.: 17/029,295

(22) Filed: Sep. 23, 2020

(30) Foreign Application Priority Data
Oct. 7, 2019 (TW) 108136226

(51) Int. Cl. A61B 5/046; A61B 5/024; A61B 5/0402; A61B 5/0456

(52) U.S. Cl. CPC A61B 5/046; A61B 5/0456; A61B 5/04028; A61B 5/02416

(57) ABSTRACT

The present disclosure provides a heart-rhythm signal-processing method, including the following steps: obtain PPG signals; perform a labeling operation to determine whether inappropriate signal data exist in the PPG signals and label the inappropriate signal data; perform a first separation operation to remove the inappropriate signal data from the PPG signals and separate the PPG signals into continuous signal segments; find peak positions of the continuous signal segments; perform a second separation operation to separate the continuous signal segments into sub-signal segments according to the number of peaks required for interpretation; perform a feature-extraction operation to obtain feature values respectively corresponding to each of the sub-signal segments, wherein the feature values are relative to peak-to-peak intervals (PPI) of the sub-signal segments; perform a judgement operation to judge the feature values using a judgement model and determine whether the heart rhythm of the subject is belong to Atrial Fibrillation.

### CROSS REFERENCE TO RELATED APPLICATIONS

This application claims priority of Taiwan Patent Application No. 108136226, filed on Oct. 7, 2019, the entirety of which is incorporated by reference herein.

### BACKGROUND OF THE INVENTION

### Field of the Invention

The present invention relates to a method of heart rhythm measurement, and, in particular, to a method of heart rhythm measurement using photoplethysmography.

### Description of the Related Art

Atrial Fibrillation (AF) is a common phenomenon of arrhythmia cordis, and the incidence of Atrial Fibrillation increases with age. However, not all people with potential Atrial Fibrillation experience the phenomenon of Atrial Fibrillation, and some people who suffer from persistent Atrial Fibrillation become used to the phenomenon. For those who have not detected or become used to Atrial Fibrillation, the importance of early medical intervention is often ignored. They don't realize it until it is too late and serious complications occur.

In the current technique for detecting arrhythmia cordis using static signals of photoplethysmography, the first step is usually to record electrocardiography (ECG) signals and static signals of photoplethysmography at the same time, and then use the ECG signals as ground truth to label whether the static signals of photoplethysmography are arrhythmia cordis. The signals of photoplethysmography will be preprocessed to find the location of each peak, and under the condition of a fixed number of peaks (usually at least 80 peaks are required), the feature values required for detection are calculated. Then, the calculated feature values and the existing classification method are used to establish the arrhythmia cordis detecting model. However, although the current method can obtain good results when using static, good signals of photoplethysmography, there are still obvious limitations. In the current method, the signals of photoplethysmography must be in a static state without noise interference to obtain an ideal result. If the signals of photoplethysmography that are used are bad signals, the current method will cause a misjudgment. Therefore, the present disclosure provides a method to solve the problem of it being difficult to collect long-term static signals of photoplethysmography. This method removes bad signals of photoplethysmography automatically through certain features and reduces the number of peaks required for detecting arrhythmia cordis. Consequently, this method is easy to perform daily.

### BRIEF SUMMARY OF THE INVENTION

An embodiment of the present disclosure provides a heart-rhythm signal-processing method, comprising the following operations: obtain photoplethysmography (PPG) signals, wherein the PPG signals are obtained from a subject through a PPG signal sensor. Perform a labeling operation to determine whether there is inappropriate signal data in the PPG signals, and to label the inappropriate signal data. Perform a first separation operation to remove the inappropriate signal data from the PPG signals, and to separate the PPG signals into continuous signal segments by using the location of the inappropriate signal data as a reference point. Find peak locations of the continuous signal segments. Perform a second separation operation to separate the continuous signal segments into sub-signal segments according to the number of peaks required for interpretation, wherein the number of peaks contained in each of the sub-signal segments is equal to the number of peaks required for interpretation. Perform a feature-extraction operation to obtain feature values that respectively correspond to the sub-signal segments, wherein the feature values are relative to the peak-to-peak intervals (PPI) of the sub-signal segments. Perform a judgement operation to judge the feature values through a judgement model that has been pre-established, and to determine whether the heart rhythm of the subject exhibits Atrial Fibrillation according to a result of the judgement. The establishment of the judgement model uses first-type feature values and second-type feature values to perform machine learning with classification algorithm to find a decision boundary that can separate the first-type feature values from the second-type feature values. Wherein the first-type feature values have been classified as belonging to Atrial Fibrillation and the second-type feature values have been classified as belonging to non-Atrial Fibrillation. The judgement model determines whether a classification of a to-be-classified feature value is Atrial Fibrillation according to the decision boundary.

An embodiment of the present invention provides an electronic device for measuring heart rhythm, the electronic device comprises an input device, a processing device, and a storage device. The input device is configured to obtain photoplethysmography (PPG) signals of a subject, wherein the PPG signals are obtained through a PPG signal sensor worn by the subject. The storage device is configured to store a program, wherein the program contains a judgement model that has been pre-established. When the program is performed by the processing device, the electronic device will perform the following operations. Perform a labeling operation to determine whether there is an inappropriate signal data in the PPG signals, and to label the inappropriate signal data. Perform a first separation operation to remove the inappropriate signal data from the PPG signals, and to separate the PPG signals into continuous signal segments by using the location of the inappropriate signal data as a reference point. Perform a signal processing operation to execute baseline removal and smoothing on the continuous signal segments, and to find the peak locations of the continuous signal segments. Perform a second separation operation to separate the continuous signal segments into sub-signal segments according to the number of peaks required for interpretation, wherein the number of peaks contained in each of the sub-signal segments is equal to the number of peaks required for interpretation. Perform a filtration operation, wherein the filtration operation obtains filtering features that respectively correspond to the sub-signal segments, determines whether each of the sub-signal segments is a bad signal according to the filtering features, and deletes sub-signal segments determined as bad signals, wherein the sub-signal segments that have not been deleted are called good sub-signal segments. Perform a feature-extraction operation to obtain feature values that respectively correspond to the good sub-signal segments, wherein the feature values are relative to peak-to-peak intervals (PPI) of the sub-signal segments. Perform a judgement operation to judge the feature values through the judgement model that has been pre-established, and to determine whether a heart rhythm of the subject belongs to Atrial Fibrillation according to a result of the judgement.

In addition, an embodiment of the present invention provides a computer program product, when the computer program product is loaded by a computer, the computer performs the following operations. Perform an obtaining operation to obtain photoplethysmography (PPG) signals, wherein the PPG signals are obtained by sensing a subject using a PPG signal sensor. Perform a labeling operation to determine whether there is inappropriate signal data in the PPG signals, and to label the inappropriate signal data. Perform a first separation operation to remove the inappropriate signal data from the PPG signals, and to separate the PPG signals into continuous signal segments by using the location of the inappropriate signal data as a reference point. Perform a signal processing operation to execute baseline removal and smoothing on the continuous signal segments, and to find peak locations of the continuous signal segments. Perform a second separation operation to separate the continuous signal segments into sub-signal segments according to the number of peaks required for interpretation, wherein the number of peaks contained in each of the sub-signal segments is equal to the number of peaks required for interpretation. Perform a filtration operation, wherein the filtration operation obtains filtering features that respectively correspond to the sub-signal segments, determines whether each of the sub-signal segments is a bad signal according to the filtering features, and deletes sub-signal segments determined as bad signals, wherein the sub-signal segments that have not been deleted are called good sub-signal segments. Perform a feature-extraction operation to obtain feature values that respectively correspond to the good sub-signal segments, wherein the feature values are relative to peak-to-peak intervals (PPI) of the sub-signal segments. Perform a judgement operation to judge the feature values through a judgement model that has been pre-established, and to determine whether a heart rhythm of the subject belongs to Atrial Fibrillation according to a result of the judgement.

### BRIEF DESCRIPTION OF THE DRAWINGS

The present disclosure is best understood from the following detailed description when read with the accompanying figures. It is emphasized that, in accordance with the standard practice in the industry, various features are not drawn to scale and are used for illustration purposes only. In fact, the dimensions of the various features may be arbitrarily increased or reduced for clarity of discussion. It is also emphasized that the drawings appended illustrate only typical embodiments of this disclosure and are therefore not to be considered limiting in scope, for the disclosure may apply equally well to other embodiments.

FIG. 1 shows a method for determining whether the heart rhythm of a subject belongs to Atrial Fibrillation based on photoplethysmography signals, in accordance with some embodiments of the present disclosure.

FIGS. 2A and 2B show diagrams of continuous signal segments of photoplethysmography signals, in accordance with some embodiments of the present disclosure.

FIGS. 3A to 3D show diagrams of processing the continuous signal segments by spline, in accordance with some embodiments of the present disclosure.

FIG. 4A shows a diagram of peak-to-peak intervals of sub-signal segments, in accordance with some embodiments of the present disclosure.

FIG. 4B shows a diagram of amplitude feature of the sub-signal segments, in accordance with some embodiments of the present disclosure.

FIG. 4C to 4D show diagrams of frequency-domain feature of the sub-signal segments, in accordance with some embodiments of the present disclosure.

FIG. 5 shows an exemplary device for performing the method shown in FIG. 1, in accordance with some embodiments of the present disclosure.

### DETAILED DESCRIPTION OF THE INVENTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

Further, spatially relative terms, such as “beneath,” “below,” “lower,” “above,” “upper” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly.

Still further, unless specifically disclaimed, the singular includes the plural and vice versa. And when a number or a range of numbers is described with “about,” “approximate,” and the like, the term is intended to encompass numbers that are within a reasonable range including the number described, such as within +/−10% of the number described or other values as understood by person skilled in the art.

In addition, the present disclosure is not limited by the illustrated ordering of acts or events, as some acts may occur in different orders and/or concurrently with other acts or events. Furthermore, not all illustrated acts or events are required to implement a methodology in accordance with the present disclosure.

The present disclosure provides a method for achieving the goal that detects Atrial Fibrillation in daily life. The method uses wearable device to measure photoplethysmography (PPG, for the purpose of simplify, photoplethysmography will be referred as PPG signal in the following description), and analyzes the PPG signal through an Atrial Fibrillation judgement model established for the method.

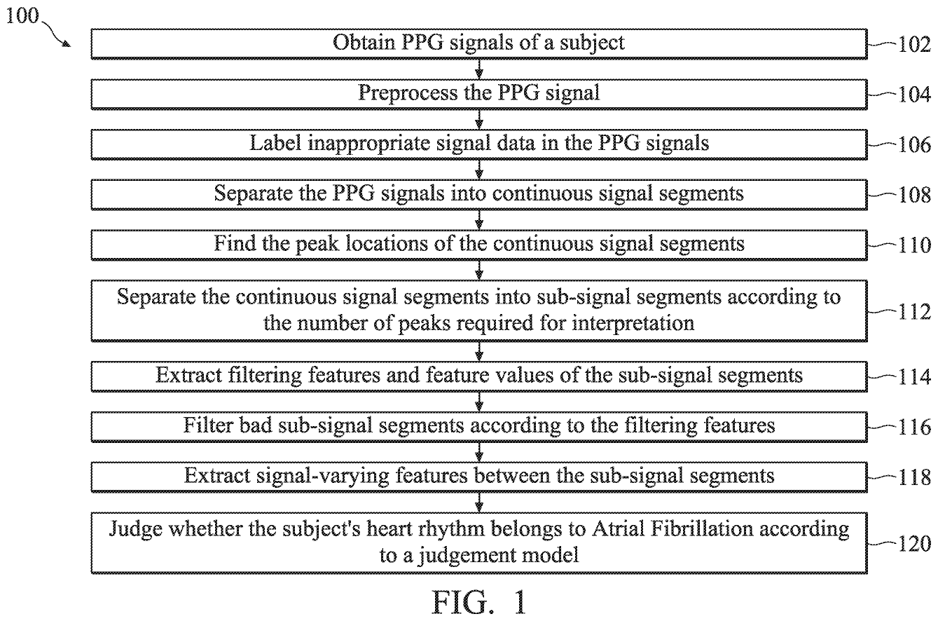

FIG. 1 shows a method 100 for determining whether the heart rhythm of a subject is Atrial Fibrillation according to the PPG signal, in accordance with some embodiments of the present disclosure.

In step 102, the method 100 obtains PPG signals of a subject. The PPG signals of the subject are obtained by sensing the subject through a PPG signal sensor. In some embodiments, the PPG sensor is a reflective sensor disposed on a wearable device, and the wearable device can be disposed on wrist, arm, forehead, or other suitable body parts for sensing PPG signals of the subject. In other embodiments, the PPG sensor is a transmission sensor, the transmission sensor senses PPG signals of the subject from fingers or other suitable body parts of the subject.

In step 104, the method 100 may perform preprocessing on the obtained PPG signals to make the subsequent steps proceed smoothly. For example, the method 100 can reorder the obtained PPG signals according to time sequence. Thereby, it ensures that the PPG signals after preprocessing are arranged according to the sensing time, not according to the obtained time.

In step 106, the method 100 may performs a labeling operation to label inappropriate signal data in the PPG signals. In some embodiments, the labeling operation comprises inspecting whether the PPG signals have signal loss. If the PPG signals have signal data loss, the labeling operation labels the signal segment having signal data loss in the PPG signals as inappropriate signal data. For example, the labeling operation inspects the PPG signals in one unit of time (e.g. 1 second (s), 2 s, or other suitable time). If the signal loss within one unit of time is more than a predetermined percentage, the PPG signals within this one unit of time are labeled as inappropriate signal data, and the data within this one unit of time will not be used in subsequent steps. The predetermined percentage can be set according to requirements, such as 3%, 5%, 10% or other suitable values. For example, assuming that one unit of time is 1 s, the sensing frequency of the PPG signal sensor is 100 times per second (i.e. the obtained PPG signals should contain 100 sets of data per second), and the predetermined percentage is 5%, when inspecting the PPG signals, if it is found that more than 5 sets of data cannot be found within one second, it is determined that the PPG signals of that second is loss, and the PPG signals of that second are labeled as inappropriate signal data.

In some embodiments, the labeling operation further comprises inspecting whether the PPG signals have signal saturation. If it finds that the PPG signals have signal saturation, the labeling operation labels the signal segment having signal saturation in the PPG signals as inappropriate signal data. The signal saturation of the PPG signals means that the values of obtained PPG signals exceed the maximum or minimum value that can be sensed by the used PPG signal senor. For example, if the maximum value and the minimum value of the used PPG signal sensor are 1500 mV and −1500 mV, respectively, the signal segment having a value higher than 1500 mV or lower than −1500 mV in the PPG signals will be regarded as signal saturation. The labeling operation inspects the PPG signals in one unit of time (e.g. 1 s, 2 s, or other suitable time). If the PPG signals in one unit of time have signal saturation, the PPG signals within this one unit of time are labeled as inappropriate signal data. Since the saturation of the signal will not disappear immediately, the PPG signals of the following two units of time will also be labeled as inappropriate signal data, and the data of this one unit of time and the following two units of time will not be used in subsequent steps.

In some embodiments, the labeling operation further comprises inspecting whether the PPG signals are static signal. If it finds that a signal segment within the PPG signal is non-static, the labeling operation labels the signal segment being non-static within the PPG signal as inappropriate signal data. For example, a three-axis accelerometer can be disposed on the PPG signal sensor for detecting whether the PPG signal sensor is in a dynamic state (i.e. detecting whether the body part where the subject is wearing the PPG signal sensor is in dynamic state). The labeling operation inspects the PPG signals in one unit of time (e.g. 1 s, 2 s, or other suitable time). If the three-axis accelerometer shows that in one unit of time, a signal segment of the PPG signals is in dynamic state, then it determines that the signal segment of the PPG signal within this one unit of time is non-static. The labeling operation labels the signal segment of the PPG signal within this one unit of time as the inappropriate signal data, and the data of this one unit of time will not be used in subsequent steps.

In step 108, the method 100 may perform a first separation operation to the PPG signals undergone the labeling operation. The first separation operation separates the PPG signals into a plurality of continuous signal segments. In the first separation operation, the portion labeled as inappropriate signal data during the step 106 will be removed from the PPG signals. The remaining of the PPG signals are separated into continuous signal segments by using the location of the inappropriate signal data as a reference point.

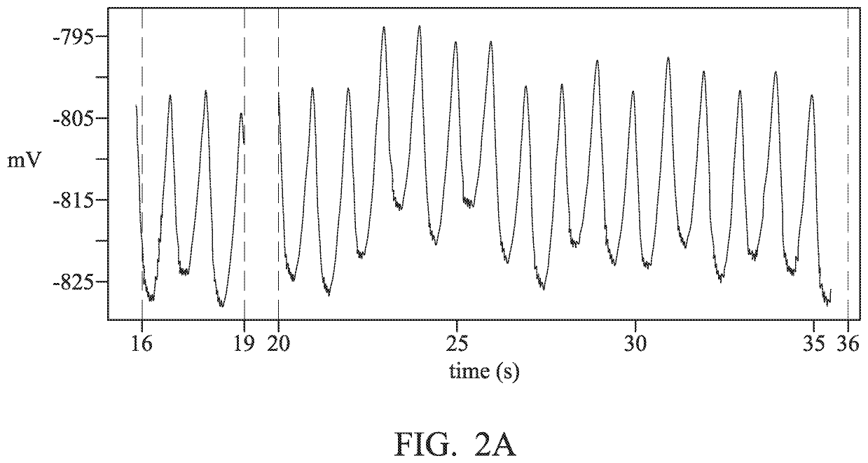

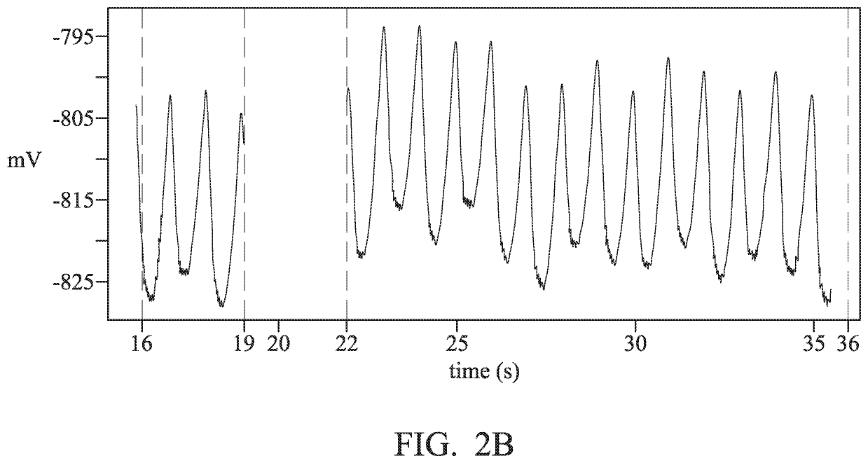

Take a 20-second PPG signal segment from the 16^{th} to the 36^{th} second as an example, and assume that one unit of time is 1 second. If the 19^{th} to 20^{th} second is labeled as inappropriate signal data due to the signal loss or being in non-static state, the signal from 19^{th} to 20^{th} second will be removed, and two continuous signal segments, 16^{th} to 19^{th} second and 20^{th} to 36^{th} second, are remained, as shown in FIG. 2A. If the 19^{th} to 20^{th} second is labeled as inappropriate signal data due to the signal saturation, the signal from 19^{th} to 22^{th} second will be removed, and two continuous signal segments, 16^{th} to 19^{th} second and 22^{th} to 36^{th} second, are remained, as shown in FIG. 2B. It should be noted that if a PPG signal segment has no inappropriate signal data, this PPG signal segment is a continuous signal segment.

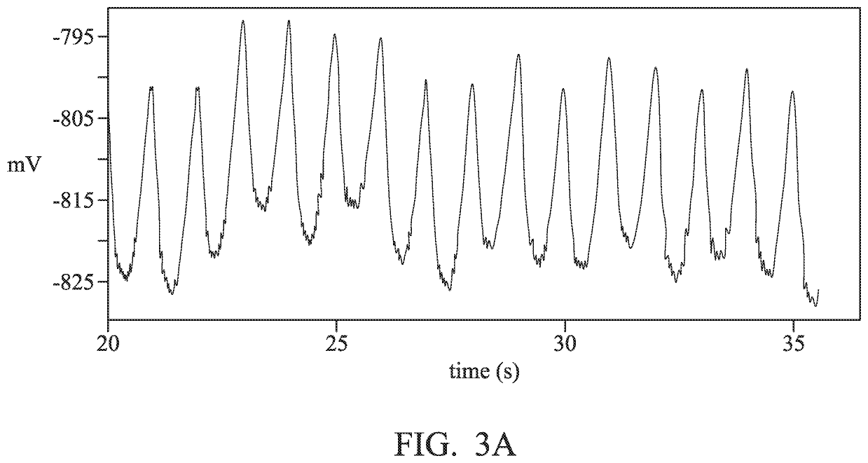

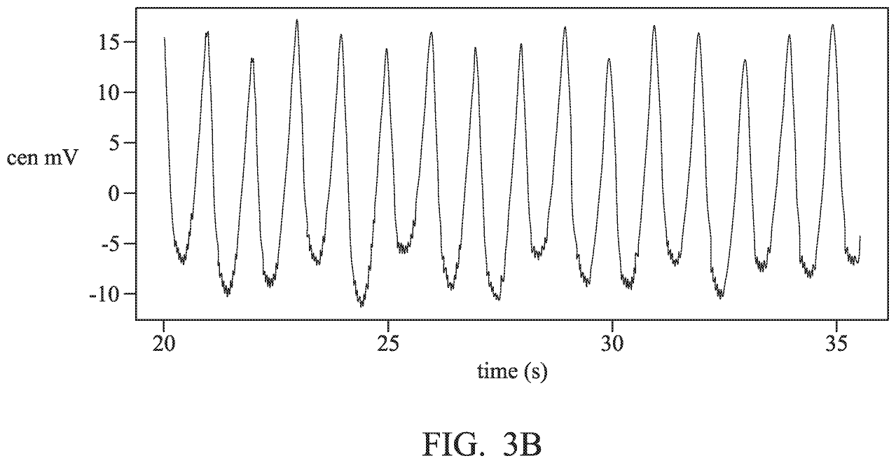

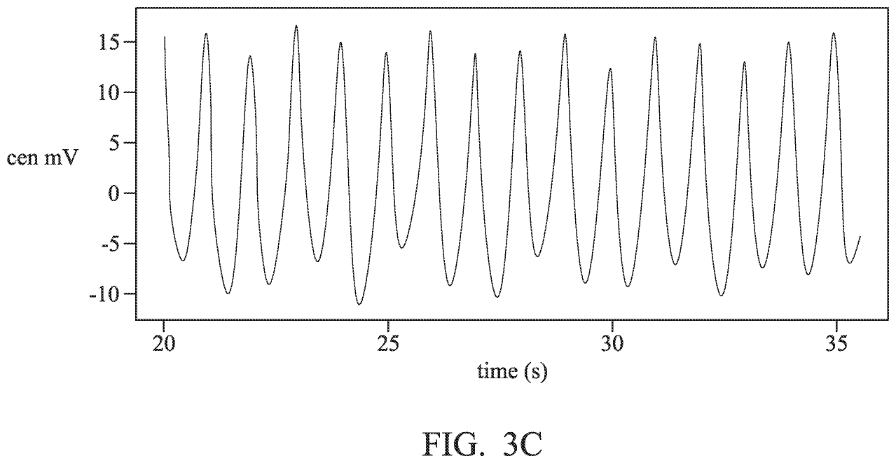

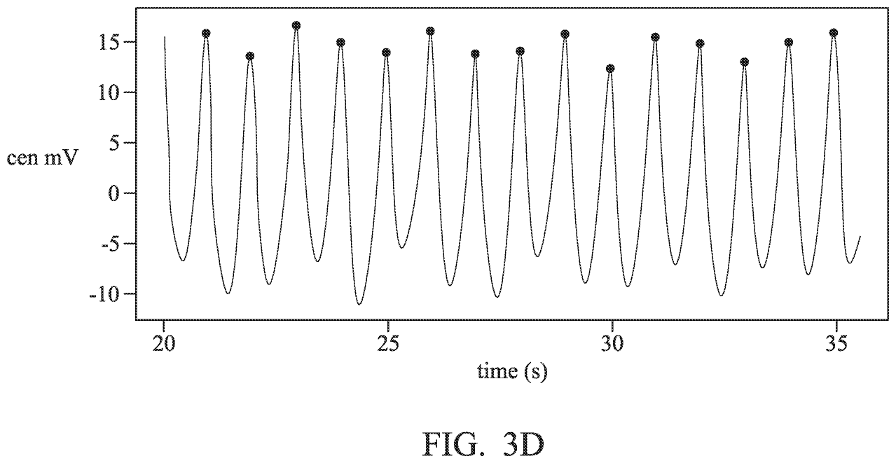

In step 110, the method 100 may find peak (wave peak) locations of each of the continuous signal segments, wherein the continuous signal segments are obtained from the step 108. In some embodiments, the step 110 uses spline to find the peak locations of each of the continuous signal segments. In some embodiments, before finding the peak locations, the method 100 first performs baseline removal and smoothing processing to each of the continuous signal segments by spline, and then finds the peak locations, as shown in FIG. 3A to 3D. FIG. 3A shows a continuous signal segment without being processed by spline. FIG. 3B shows the continuous signal segment after baseline removal. FIG. 3C shows the continuous signal segment after smoothing processing. FIG. 3D shows the continuous signal segment after the peak locations being found. In other embodiments, the baseline removal and the smoothing processing may be performed by other methods, such as smoother, Fourier transform, and Hilbert-Huang Transform, but not limited thereto.

In step 112, the method 100 may perform a second separation operation to separate the continuous signal segments into sub-signal segments according to the number of peaks required for interpretation, wherein the continuous signal segments are obtained from the step 110. The number of peaks required for interpretation presents the number of peaks contained in each of the sub-signal segments, wherein the sub-signal segments are used to determine whether the heart rhythm of the subject is Atrial Fibrillation in the subsequent steps of the method 100. In some embodiments, the number of peaks required for interpretation is 10, but not limited thereto.

In some embodiments, the continuous signal segment whose number of peaks is lower than the number of peaks required for interpretation will be removed. The continuous signal segment whose number of peaks is higher than the number of peaks required for interpretation will be separated into sub-signal segments according to the number of peaks required for interpretation. In certain embodiments, for the continuous signal segment whose number of peaks is higher than the number of peaks required for interpretation but cannot be divisible by the number of peaks required for interpretation, after separating the last complete sub-signal segment, a sub-signal segment whose number of peaks is equal to the number of peaks required for interpretation will be taken out from the end of the continuous signal segment. Take FIG. 3D as an example (the continuous signal segment of FIG. 3D has 15 peaks) and assume that the number of peaks required for interpretation is 10, after separating a complete sub-signal segment (peak 1˜peak 10), there will be 5 peaks left in the continuous signal segment of FIG. 3D. At this time, the method 100 may take the last 10 peaks (peak 6 to peak 15) as another sub-signal segment.

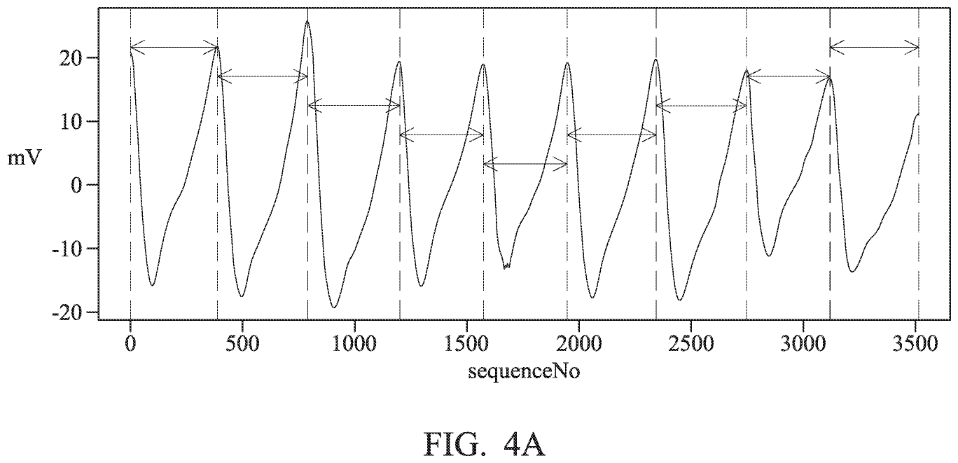

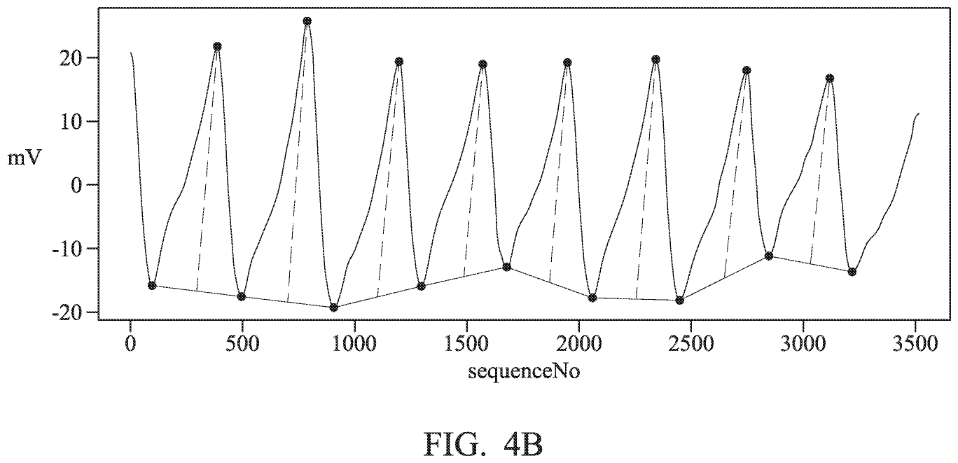

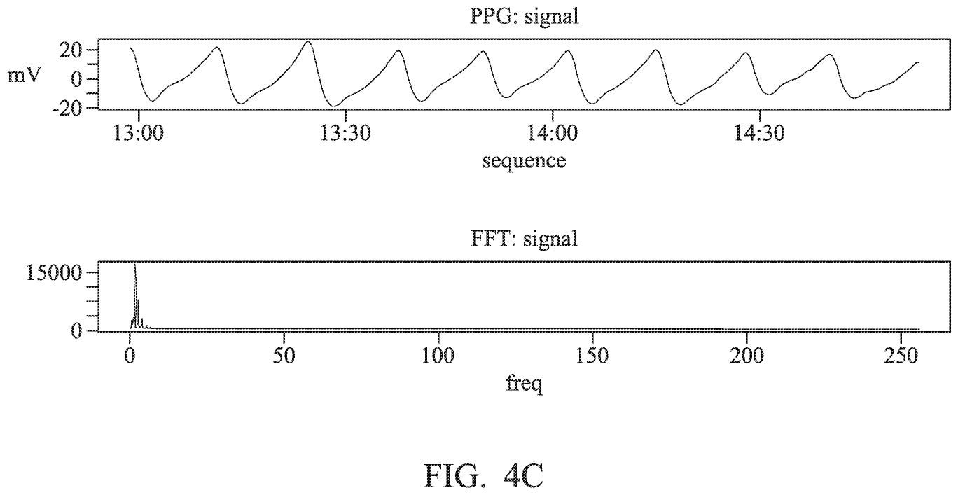

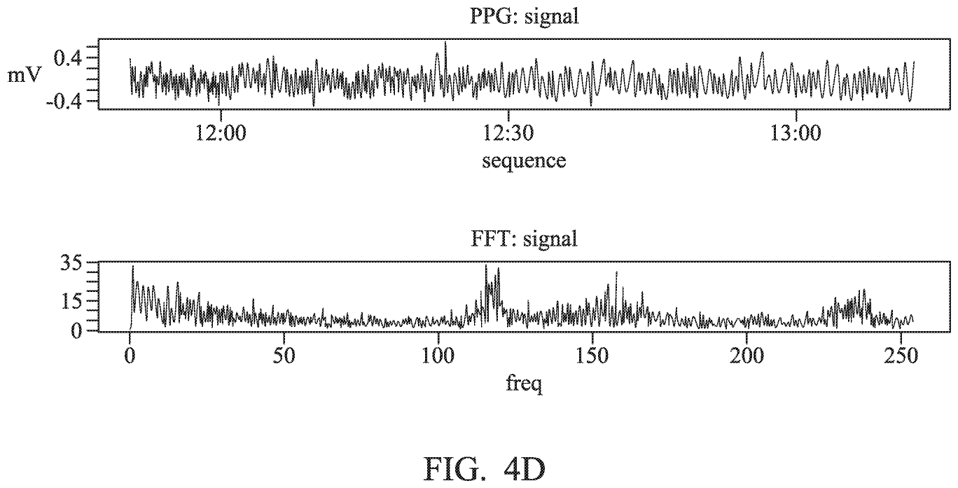

In step 114, the method 100 may extract filtering features and feature values from each of the sub-signal segments. The filtering features are used to determine whether each of the sub-signal segments is bad signal. The filtering features comprise heartbeat interval feature, amplitude feature, and frequency-domain feature. The heartbeat interval feature may be peak-to-peak interval (PPI) of the sub-signal segment, as shown in FIG. 4A. Because the interval of human heartbeats will be within a reasonable range, there is a set of threshold values for heartbeat interval feature in step 114. When the heartbeat interval feature exceeds the range of threshold values, it means that there is a problem within the sub-signal segment, and the method 100 will determine the sub-signal segment as a bad signal.

The amplitude feature may be the length of the connection between the peak and the midpoint of two adjacent troughs of the sub-signal segment, as shown in FIG. 4B. Similar to the heartbeat interval feature, because the amplitude of human heartbeats will be within a reasonable range, there is a set of threshold values for amplitude feature in step 114. When the amplitude feature exceeds the range of threshold values, it means that there is a problem within the sub-signal segment, and the method 100 will determine the sub-signal segment as a bad signal.

The frequency-domain feature may be used to determine whether the PPG signal sensor is normally worn by the subject. When it is determined that the PPG signal sensor is not normally worn by the subject, it means that the obtained PPG signal cannot correctly reflect the heart rhythm of the subject. Therefore, the sub-signal segment also belongs to bad signal. When the PPG signal sensor is worn normally, the main frequency of the signal will be at the heartbeat frequency, as shown in FIG. 4C. When the general PPG signal is converted to the frequency domain by Fast Fourier Transform (FFT), the main frequency representing the heartbeat will appear. If the PPG signal sensor is not worn normally, the frequency of the PPG signal obtained at this time will be evenly dispersed after Fast Fourier Transform, as shown in FIG. 4D. Because there is a reasonable range of human heartbeat frequency, it may set a particular frequency, and signals beyond the particular frequency are regarded as non-heartbeat signals. The particular frequency may be 10 Hertz (Hz), but not limited thereto. The frequency-domain feature may be defined as: (the sum of the intensity greater than the particular frequency)/(the sum of the intensity of all frequencies). The larger the value of the frequency-domain feature, the higher the ratio of non-heartbeat signals in the signal. Therefore, there is a set of threshold values for frequency-domain feature in step 114. When the frequency-domain feature is greater than the threshold value, it means that the source of the sub-signal segment is a PPG signal sensor that is not normally worn, and the method 100 will determine the sub-signal segment as a bad signal.

In the subsequent steps, the feature values will be used to determine whether the heart rhythm of the subject belongs to Atrial Fibrillation. The feature values comprise peak-to-peak interval Standard Deviation (PPI SD), peak-to-peak interval Root Mean Square Successive Differences (PPI RMSSD), and peak-to-peak Entropy (PPI Entropy).

The PPI SD may be defined as:

$$PPI SD = \sqrt{\frac{1}{n - 1} {{Σ}_{i = 1}^{n} ( {PPI}_{i} - \overline{PPI} )}^{2}}$$

Wherein n is the total number of the required PPI. For example, when the number of peaks required for interpretation is 10, the n is equal to 9. The PPI RMSSD may be defined as:

$$PPI RMSSD = \sqrt{\frac{1}{n} {{Σ}_{i = 2}^{n} ( {PPI}_{i} - {PPI}_{i - 1} )}^{2}}$$

The PPI Entropy may be defined as:

PPI Entropy=Σᵢ₌₁^{k} [symbol] log( [symbol] )

When using the minimum and maximum values of n PPIs to cut into k segments, [symbol] is the sample probability value of the i-th segment.

It should be noted that the filtering features comprise heartbeat interval feature, amplitude feature, and frequency-domain feature, and the feature values comprise PPI SD, PPI RMSSD, and PPI Entropy, but the present disclosure is not limited thereto. Those skilled in the art may readily add, replace, or eliminate these features, and these variations are encompassed by the present disclosure.

In step 116, the method 100 may perform a filtration operation. In the filtration operation, the method 100 removes the sub-signal segments determined as bad signal in step 114. These removed sub-signal segments will not enter the subsequent steps of the method 100.

In some embodiments, the method 100 may perform step 116 before performing the extraction of feature values in step 114. In these embodiments, the method 100 will first remove the sub-signal segments determined as bad signal after extracting the filtering features and determining which sub-signal segments are bad signals, and then perform the extraction of feature values on the remaining sub-signal segments.

In step 118, the method 100 may extract signal-varying features between sub-signal segments according to the feature values of each of the sub-signal segments. The signal-varying features may comprise Standard Deviation-varying feature related to peak-to-peak interval (diff(PPI SD)), Peak-to-Peak Interval Root Mean Square Successive Differences-varying feature (diff(PPI RMSSD)), sub-signal segment max intensity frequency-varying feature (diff(max Freq)), the average value or difference between the same feature values of different sub-signal segments and a like, but not limited thereto.

In some embodiments, the extraction of the signal-varying features is to set a reference segment number S first, wherein S is a positive integer greater than or equal to 1. When the (S+1)^{th} sub-signal segment is selected as a judgement sub-signal segment for the subsequent steps of the method 100, signal-varying features are extracted based on each of the feature values of the (S+1)^{th} sub-signal segment and previous S sub-signal segments (total S+1 sub-signal segments). These signal-varying features are used in subsequent steps of the method 100. For example, if the reference segment number is set as 2 and the 3^{rd} sub-signal segment is selected as judgement sub-signal segment for subsequent steps, each of the feature values of the 3^{rd} sub-signal segment and the previous two segments will be used to extract signal-varying features. On the other word, each of the feature values of the 1^{st}, 2^{nd} and 3^{rd} sub-signal segments will be used to extract signal-varying features.

The Standard Deviation-varying feature related to peak-to-peak interval (diff(PPI SD)) may be defined as:

$$diff ( PPI SD ) = \frac{1}{s + 1} {\sum }_{i = 1}^{s + 1} {( PPI SD )}_{i}$$

Wherein s is the reference segment number, and (s+1) is the total number of segments that reference segment number plus the judgement sub-signal segment. The Peak-to-Peak Interval Root Mean Square Successive Differences-varying feature (diff(PPI RMSSD)) may be defined as:

$$diff ( PPI RMSSD ) = \sqrt{\frac{1}{s + 1} {{Σ}_{i = 2}^{s + 1} ( {( PPI RMSSD )}_{i} - {( PPI RMSSD )}_{i - 1} )}^{2}}$$

The sub-signal segment max intensity frequency-varying feature (diff(max Freq)) may be defined as:

$$diff ( max Freq ) = \sqrt{\frac{1}{s + 1} {{Σ}_{i = 2}^{s + 1} ( {( max Freq )}_{i} - {( max Freq )}_{i - 1} )}^{2}}$$

Wherein (max Freq)ᵢ is the frequency of maximum intensity of the i-th signal segment.

In step 120, the method 100 judges whether the subject's heart rhythm belongs to Atrial Fibrillation according to a judgement model established in advance with a classification algorithm. In some embodiments, the judgement model performs judgement based on the signal-varying features obtained in step 118. For example, if the reference segment number is set as 2 and the 3^{rd} sub-signal segment is selected as judgement sub-signal segment, the judgment model judges whether the heart rhythm belongs to Atrial Fibrillation based on the signal-varying features extracting from each of the feature values of the 1^{st} to 3^{rd} sub-signal segments.

In some embodiments, in addition to the signal-varying features obtained in step 118, the judgment model also uses feature values at the same time for judgement. For example, if the reference segment number is set as 2 and the 3^{rd} sub-signal segment is selected as judgement sub-signal segment, in addition to the signal-varying features extracted from the feature values of the 1^{st} to 3^{rd} sub-signal segments, the judgment model will also use the feature values of the 3^{rd} sub-signal segment to perform judgement. In addition, the judgment model may also use the feature values of the 1^{st} and/or 2^{nd} sub-signal segments at the same time to perform judgement.

In some embodiments, the method 100 may skip step 118 and directly use the feature values obtained in step 114 (which have undergone the filtering operation of step 116) to perform judgement. For example, if the 3^{rd} sub-signal segment is selected as judgement sub-signal segment, the judgement model can directly use the feature values of the 3^{rd} sub-signal segment to perform judgement.

It should be noted that, since the performing of step 108 (the first separation operation), step 112 (separating the continuous signal segments into sub-signal segments), and step 116 (filtering the bad sub-signal segments), each of the sub-signal segments may be continuous or discontinuous with each other.

The establishment of the judgment model is based on the following method: obtain data features corresponding to each of observable events and known result labels corresponding to each of the observable events from the observable events, wherein each of the known result labels is one of two known classifications; use a classification algorithm to perform machine learning based on observable events, and data features and known result labels corresponding to each of the observable events to find a decision boundary, and then establish the judgement model, wherein the decision boundary can distinguish the data features corresponding to different known result labels. In this way, if there are new events of the same type as the observable events, the judgement model can judge which of the two known classifications the new events belong to according to the decision boundary and the data features corresponding to the new events.

For example, observable events are PPG signals of precedent subjects; data features are feature values and signal-varying features corresponding to the PPG signals of the precedent subjects; and the two known classifications are Atrial Fibrillation and non-Atrial Fibrillation. In other words, the establishment of the judgement model is to first collect a large number of PPG signals known as Atrial Fibrillation or non-Atrial Fibrillation from different precedent subjects, and data of feature values and/or signal-varying features corresponding to these PPG signals. Perform machine learning on this data with classification algorithms to find a decision boundary, and then establish the judgement model. The decision boundary can separate the feature values and/or signal-varying features corresponding to Atrial Fibrillation from the feature values and/or signal-varying features corresponding to non-Atrial Fibrillation. In this way, the judgement model can classify the PPG signals of the subjects as Atrial Fibrillation or non-Atrial Fibrillation according to the decision boundary and the feature values and/or the signal-varying features of the PPG signals of the subjects. After the judgement model is established, if there are PPG signals of a subject that needs to be judged, just input the feature values and/or signal-varying features corresponding to the PPG signals, and the judgement model can judge that the PPG signals belong to Atrial Fibrillation or non-Atrial Fibrillation. The classification algorithms described above include support vector machine (SVM), deep learning, XGBoost, random forest, and/or other suitable algorithms.

It should be noted that different judgment models that use different feature values and/or signal-varying features for judgement have different machine learning processes. For example, in an embodiment where the reference segment number is 2 and 3 types of signal-varying features are used, the machine learning of judgement model is performed based on 3 types of signal-varying features of a total of 3 sub-signal segments. If the embodiment uses 4 types of signal-varying features, the machine learning of judgement model is performed based on 4 types of signal-varying features of a total of 3 sub-signal segments. For example, in an embodiment where the reference segment number is 2 and three signal-varying features are used, if three feature values of the judgement sub-signal segments are additionally used in this embodiment, these three feature values should be added into the machine learning process of the judgment model. To take a more specific example, in an embodiment that only uses the PPI SD and PPI RMSSD of the judgment sub-signal segment, the machine learning of the judgment model is performed based on the PPI SD and PPI RMSSD of a single sub-signal segment.

In addition, if the number of peaks required for interpretation is different, the machine learning process of the judgment model is also different. For example, if the number of peaks required for interpretation is 10, the number of peaks contained in sub-signal segment used for machine learning of judgement model is 10; and if the number of peaks required for interpretation is 15, the number of peaks contained in sub-signal segment used for machine learning of judgement model is 15.

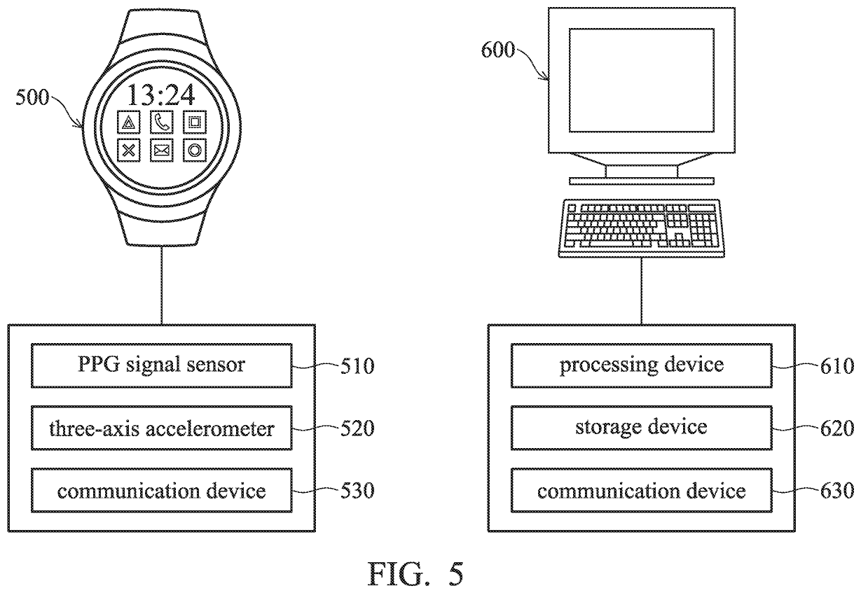

FIG. 5 shows an exemplary device according to some embodiments of the present disclosure. The exemplary device may comprise a wearable device 500 and a computing device 600. The wearable device 500 comprises a PPG signal sensor 510, a three-axis accelerometer 520, and communication device 530. The computing device 600 comprises a processing device 610, a storage device 620, and a communication device 630.

The wearable device 500 may be directly worn by the subject. For example, the wearable device 500 may be worn on the wrist, arm, forehead, or other suitable body parts. The PPG signal sensor 510 is used to sense PPG signals of the subject. While the PPG signal sensor 510 is operating, the three-axis accelerometer 520 can simultaneously detect whether the PPG signal sensor 510 is in a non-static state. It means that the three-axis accelerometer 520 can detect whether the part of the subject wearing the wearable device 500 is in dynamic state. The communication device 530 of the wearable device 500 can be connected with the communication device 630 of the computing device 600 to transmit the data obtained by the PPG signal sensor 510 and the three-axis accelerometer 520 to the computing device 600.

The storage device 620 may store a program including the method 100 and the judgement model described above, and the processing device 610 may execute the program to implement the method 100. When the communication device 630 of the computing device 600 receives the data from the wearable device 500, the processing device 610 can execute the program stored in the storage device 620 to process the data according to the method 100, and finally judge whether the subject's heart rhythm is Atrial Fibrillation according to the judgement model in the method 100. The data from the wearable device 500 comprises the data sensed by the PPG signal sensor 510 and the data detected by the three-axis accelerometer 520.

In some embodiments, the communication device 530 and the communication device 630 may be wirelessly and/or wired to communicate with each other. In some embodiments, the computing device 600 may be a computer, a tablet computer, a mobile phone, a cloud server, or other devices with processing functions. In some embodiments, the wearable device 500 and the computing device 600 may be integrated together, for example, integrated into a single wearable device.

The method 100 and the determination model described above can be implemented as a computer program product and executed by the computing device 600. The computer program product can be stored in the storage device 620, and can be loaded and performed by the processing device 610. When the computer program product is performed, the operations that can be performed include: an obtaining operation, a labeling operation, a first separation operation, a signal processing operation, a second separation operation, a filtration operation, a feature-extraction operation, a signal-varying feature-extraction operation, and a judgement operation.

The obtaining operation is used to obtain PPG signals, wherein the PPG signals are obtaining by sensing a subject using a PPG signal sensor (e.g. PPG signal sensor 510). The labeling operation is used to determine whether there is inappropriate signal data in the PPG signal in the obtained PPG signals and label the inappropriate signal data. The inappropriate signal data means: a ratio of a part lacking data in the PPG signals exceeds a predetermined percentage (e.g. 5%) within one unit of time (e.g. 1 s); in itself or within two units of time, the PPG signals have reached saturation value; and/or within one unit of time, the accelerometer (e.g. the three-axis accelerometer 520) determines that the PPG signal sensor has been in a non-static state.

The first separation operation is used to remove the inappropriate signal data from the PPG signals, and separate the PPG signals into continuous signal segments by using the location of the inappropriate signal data as a reference point. The signal processing operation is used to execute baseline removal and smoothing on the continuous signal segments, and find the peak locations of the continuous signal segments. The second separation operation is used to separate the continuous signal segments into sub-signal segments according to the number of peaks required for interpretation, wherein the number of peaks contained in each of the sub-signal segments is equal to the number of peaks required for interpretation.

The filtration operation is used to obtain filtering features that respectively correspond to the sub-signal segments. The filtration operation is also used to determine whether each of the sub-signal segments is a bad signal according to the filtering features, and then delete the sub-signal segments determined as bad signals. Wherein the sub-signal segments that have not been deleted are called good sub-signal segments. The feature-extraction is used to obtain feature values that respectively correspond to the good sub-signal segments, wherein the feature values are relative to peak-to-peak intervals (PPI) of the sub-signal segments. The judgement operation is used to judge the feature values through a judgement model, and determine whether the heart rhythm of the subject belongs to Atrial Fibrillation according to a result of the judgement.

The signal-varying feature-extraction operation is used to obtain signal-varying features. The signal-varying feature-extraction operation comprises following steps: obtain the feature values of a particular good sub-signal segment in the good sub-signal segments; obtain the feature values of N good sub-signal segments before the particular good sub-signal segment from the good sub-signal segments, wherein N is a positive integer greater than or equal to 1; obtain signal-varying features according to the feature values of the particular good sub-signal segment and the feature values of the N good sub-signal segments. It should be noted that, when the judgment operation performs judgement through the judgment model, in addition to using feature values, signal-varying features may also be used, or both feature values and signal-varying features may be used at the same time. In other words, the judgment operation can use the judgment model to judge feature values, signal-varying features, or combinations thereof, and determine whether the heart rhythm of the subject belongs to Atrial Fibrillation.

The various embodiments or examples in the foregoing description illustrate various advantages of the present disclosure. In method 100, step 106 labels the inappropriate signal data in the PPG signals; step 108 removes the inappropriate signal data; step 114 extracts the filtering features of the sub-signal segments, and determines whether each of the sub-signal segment is bad signal; and step 116 filters out the sub-signal segments determined as bad signals. Through these steps, the present disclosure can ensure that all of sub-signal segments that finally use for judgement are PPG signal with good quality. As a result, the misjudgement of the judgement model due to the PPG signals with poor quality (e.g. interfered by noise or motion) can be avoided.

In addition, the number of peaks contained in the sub-signal segments of the PPG signals used in the present disclosure is far fewer than the number of peaks that prior art needed. It means that the prior art requires a long-term static PPG signal to operate. However, the static duration required for the sub-signal segments of the PPG signals used in the present disclosure is far less than the static duration required by the prior art. Therefore, in daily life, the wearable PPG signal sensor can easily collect the sub-signal segments of the PPG signals required by the present disclosure without that the subject has to keep static deliberately.

Furthermore, by setting the reference segment number and simultaneously using the feature values and/or signal-varying features of a plurality of sub-signal segments with good quality for judgement, the present disclosure can effectively improve the accuracy of Atrial Fibrillation judgement. And as described above, the static duration required for the sub-signal segments of the PPG signals used in the present disclosure is far less than the static duration required by the prior art. Therefore, in daily life, a plurality of sub-signal segments with good quality can be easily collected for use in the judgement model. Accordingly, the method provided by the embodiments of the present disclosure can be readily implemented in daily life to achieve the purpose of real-time detection of Atrial Fibrillation in daily life.

The foregoing has outlined features of several embodiments so that those skilled in the art may better understand the detailed description that follows. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions and alterations herein without departing from the spirit and scope of the present disclosure.

What is claimed is:

1. A heart-rhythm signal-processing method, comprising:
obtaining photoplethysmography (PPG) signals, wherein the PPG signals are obtained by sensing a subject using a PPG signal sensor;
performing a labeling operation to determine whether there is inappropriate signal data in the PPG signals, and to label the inappropriate signal data;
performing a first separation operation to remove the inappropriate signal data from the PPG signals, and to separate the PPG signals into continuous signal segments by using a location of the inappropriate signal data as a reference point;
finding peak locations of the continuous signal segments;
performing a second separation operation to separate the continuous signal segments into sub-signal segments according to the number of peaks required for interpretation, wherein the number of peaks contained in each of the sub-signal segments is equal to the number of peaks required for interpretation;
performing a feature-extraction operation to obtain feature values that respectively correspond to the sub-signal segments, wherein the feature values are relative to peak-to-peak intervals (PPI) of the sub-signal segments; and
performing a judgement operation to judge the feature values through a judgement model that has been pre-established, and to determine whether a heart rhythm of the subject belongs to Atrial Fibrillation according to a result of the judgement.

2. The heart-rhythm signal-processing method as claimed in claim 1, wherein the labeling operation further comprises:
inspecting whether the PPG signals have signal loss, if a ratio of a part lacking data in the PPG signals exceeds a predetermined percentage within one unit of time, it is determined that there is signal loss, and the one unit of time having signal loss is labeled as the inappropriate signal data;
inspecting whether the PPG signals have signal saturation, and if the PPG signals have achieved a saturation value within one unit of time, it is determined that there is signal saturation, and the one unit of time having signal saturation and the following two units of time are labeled as the inappropriate signal data; and
inspecting whether the PPG signals are static, to determine whether the PPG signal sensor has been in a non-static state through a three-axis accelerometer, and if the PPG signal sensor has been in the non-static state within one unit of time, the PPG signals in the one unit of time are determined as non-static signals, and the one unit of time of the non-static signals is labeled as the inappropriate signal data.

3. The heart-rhythm signal-processing method as claimed in claim 1, further comprising: performing a baseline removal and a smoothing operation on the continuous signal segments.

4. The heart-rhythm signal-processing method as claimed in claim 1, wherein the feature-extraction operation further comprises a filtration operation, the filtration operation comprising:
obtaining filtering features that respectively correspond to the sub-signal segments;
determining whether each of the sub-signal segments is a bad signal according to the filtering features; and
deleting the sub-signal segments determined as bad signals, wherein the sub-signal segments that are deleted will not enter the judgement operation.

5. The heart-rhythm signal-processing method as claimed in claim 4, wherein the filtering features include at least one of heartbeat interval feature, amplitude feature, and frequency-domain feature.

6. The heart-rhythm signal-processing method as claimed in claim 1, wherein the Square Successive Differences, and Entropy that are related to peak-to-peak intervals of the sub-signal segments.

7. The heart-rhythm signal-processing method as claimed in claim 1, further comprising a signal-varying feature-extraction operation, wherein the signal-varying feature-extraction operation comprises:
obtaining the feature values of a particular sub-signal segment among the sub-signal segments;
obtaining the feature values of N sub-signal segments before the particular sub-signal segment from the sub-signal segments, wherein N is a positive integer greater than or equal to 1;
obtaining signal-varying features according to the feature values of the particular sub-signal segment and the feature values of the N sub-signal segments; and
when the heart-rhythm signal-processing method comprises the signal-varying feature-extraction operation, the judgement operation judges the signal-varying features through the judgement model, and determines whether the heart rhythm of the subject is belong to Atrial Fibrillation.

8. The heart-rhythm signal-processing method as claimed in claim 7, wherein the signal-varying features include a least one of Standard Deviation-varying feature, Peak-to-Peak Interval Root Mean Square Successive Differences-varying feature, and sub-signal segment max intensity frequency-varying feature that are related to peak-to-peak intervals between the particular sub-signal segment and the N sub-signal segments.

9. The heart-rhythm signal-processing method as claimed in claim 1, wherein the classification algorithm is support vector machine, deep learning, XGBoost, or random forest.

10. The heart-rhythm signal-processing method as claimed in claim 1, wherein the establishment of the judgement model uses first-type feature values and second-type feature values to perform machine learning with a classification algorithm to find a decision boundary that can separate the first-type feature values from the second-type feature values, the first-type feature values have been classified as belonging to Atrial Fibrillation and the second-type feature values have been classified as belonging to non-Atrial Fibrillation, and wherein the judgement model determines whether a classification of a to-be-classified feature value belongs to Atrial Fibrillation according to the decision boundary.

11. An electronic device for measuring heart rhythm, comprising:
an input device, configured to obtain photoplethysmography (PPG) signals of a subject, wherein the PPG signals are obtained through a PPG signal sensor worn by the subject;
a processing device;
a storage device, configured to store a program, wherein the program contains a judgement model that has been pre-established, and when the program is performed by the processing device, the electronic device performs:
a labeling operation, to determine whether there is inappropriate signal data in the PPG signals, and to label the inappropriate signal data;
a first separation operation, which removes the inappropriate signal data from the PPG signals, and separates the PPG signals into continuous signal segments by using the location of the inappropriate signal data as a reference point;
a signal processing operation, which executes baseline removal and smoothing on the continuous signal segments, and finds peak locations of the continuous signal segments;
a second separation operation, which separates the continuous signal segments into sub-signal segments according to the number of peaks required for interpretation, wherein the number of peaks contained in each of the sub-signal segments is equal to the number of peaks required for interpretation;
a filtration operation, which obtains filtering features that respectively correspond to the sub-signal segments, determines whether each of the sub-signal segments is a bad signal according to the filtering features, and deletes the sub-signal segments determined as bad signals, wherein the sub-signal segments that have not been deleted are called good sub-signal segments;
a feature-extraction operation, which obtains feature values that respectively correspond to the good sub-signal segments, wherein the feature values are relative to peak-to-peak intervals (PPI) of the sub-signal segments; and
a judgement operation, which judges the feature values through the judgement model that has been pre-established, and determines whether a heart rhythm of the subject belongs to Atrial Fibrillation according to a result of the judgement.

12. The electronic device as claimed in claim 11, wherein when the program is performed by the processing device, the electronic device further performs a signal-varying feature-extraction operation, wherein the signal-varying feature-extraction operation comprises:
obtaining the feature values of a particular good sub-signal segment among the good sub-signal segments;
obtaining the feature values of N good sub-signal segments before the particular good sub-signal segment from the good sub-signal segments, wherein N is a positive integer greater than or equal to 1;
obtaining signal-varying features according to the feature values of the particular good sub-signal segment and the feature values of the N good sub-signal segments; and
when the electronic device performs the signal-varying feature-extraction operation, the judgement operation judges the signal-varying features through the judgement model, and determines whether the heart rhythm of the subject belongs to Atrial Fibrillation.

13. The electronic device as claimed in claim 12, wherein:
the filtering features include at least one of heartbeat interval feature, amplitude feature, and frequency-domain feature;
the feature values include at least one of Standard Deviation, Peak-to-Peak Interval Root Mean Square Successive Differences, and Entropy that are related to peak-to-peak intervals of the sub-signal segments; and
the signal-varying features include a least one of Standard Deviation-varying feature, Peak-to-Peak Interval Root Mean Square Successive Differences-varying feature, and sub-signal segment max intensity frequency-varying feature that are related to peak-to-peak intervals between the particular sub-signal segment and the N sub-signal segments.

14. The electronic device as claimed in claim 11, wherein the establishment of the judgement model uses first-type feature values and second-type feature values to perform machine learning with a classification algorithm to find a decision boundary that can separate the first-type feature values from the second-type feature values, the first-type feature values have been classified as belonging to Atrial Fibrillation and the second-type feature values have been classified as belonging to non-Atrial Fibrillation, and wherein the judgement model determines whether a classification of a to-be-classified feature value belongs to Atrial Fibrillation according to the decision boundary.

15. A computer program product, wherein the computer program product is loaded by a computer, and the computer performs:
an obtaining operation, which obtains photoplethysmography (PPG) signals, wherein the PPG signals are obtained by sensing a subject using a PPG signal sensor;
a labeling operation, to determine whether there is inappropriate signal data in the PPG signals, and to label the inappropriate signal data;
a first separation operation, which removes the inappropriate signal data from the PPG signals, and separates the PPG signals into continuous signal segments by using the location of the inappropriate signal data as a reference point;
a signal processing operation, which executes baseline removal and smoothing on the continuous signal segments, and finds peak locations of the continuous signal segments;
a second separation operation, which separates the continuous signal segments into sub-signal segments according to the number of peaks required for interpretation, wherein the number of peaks contained in each of the sub-signal segments is equal to the number of peaks required for interpretation;
a filtration operation, which obtains filtering features that respectively correspond to the sub-signal segments, determines whether each of the sub-signal segments is a bad signal according to the filtering features, and deletes the sub-signal segments determined as bad signals, wherein the sub-signal segments that have not been deleted are called good sub-signal segments;
a feature-extraction operation, which obtains feature values that respectively correspond to the good sub-signal segments, wherein the feature values are relative to peak-to-peak intervals (PPI) of the sub-signal segments; and
a judgement operation, which judges the feature values through a judgement model that has been pre-established, and determines whether a heart rhythm of the subject belongs to Atrial Fibrillation according to a result of the judgement.

16. The computer program product as claimed in claim 15, when the computer program product is loaded by the computer, the computer further performs a signal-varying feature-extraction operation, wherein the signal-varying feature-extraction operation comprises:
obtaining the feature values of a particular good sub-signal segment in the good sub-signal segments;
obtaining the feature values of N good sub-signal segments before the particular good sub-signal segment from the good sub-signal segments, wherein N is a positive integer greater than or equal to 1;
obtaining signal-varying features according to the feature values of the particular when the computer performs the signal-varying feature-extraction operation, the judgement operation judges the signal-varying features through the judgement model, and determines whether the heart rhythm of the subject belongs to Atrial Fibrillation.

17. The computer program product as claimed in claim 15, wherein the establishment of the judgement model uses first-type feature values and second-type feature values to perform machine learning with a classification algorithm to find a decision boundary that can separate the first-type feature values from the second-type feature values, the first-type feature values have been classified as belonging to Atrial Fibrillation and the second-type feature values have been classified as belonging to non-Atrial Fibrillation, and wherein the judgement model determines whether a classification of a to-be-classified feature value belongs to Atrial Fibrillation according to the decision boundary.
